(19) United States
(12) Patent Application Publication
(10) Pub. No.: US 2015/0308878 A1
(43) Pub. Date: Oct. 29, 2015

(54) STARTER OIL QUANTITY INDICATION SYSTEM

(71) Applicant: Hamilton Sundstrand Corporation, Charlotte, NC (US)

(72) Inventors: John T. Pech, Canton, CT (US); Benjamin T. Harder, DeKalb, IL (US)

(73) Assignee: HAMILTON SUNDSTRAND CORPORATION, Charlotte, NC (US)

(21) Appl. No.: 14/260,819

(22) Filed: Apr. 24, 2014

(51) Int. Cl. G01F 23/24; G01K 7/02; G01N 33/28; F01D 19/00; F01D 15/12

(52) U.S. Cl. CPC G01F 23/246; F01D 19/00; F01D 15/12; G01N 33/28; G01K 7/02

(57) ABSTRACT

A starter can be configured for use with a turbomachine and includes a housing defining a cavity, wherein the cavity is configured to hold a quantity of starter oil, and a temperature sensor disposed within the cavity and configured to sense a temperature within the housing. A system includes a starter for a turbomachine as described herein and a computing apparatus configured to receive signals from the temperature sensor and to determine if the temperature within the housing indicates a sufficient quantity of starter oil. A method for determining oil quantity in a starter includes receiving a temperature signal from a temperature sensor disposed within the starter, determining an oil temperature within the starter based on comparing the temperature signal, and determining if a sufficient amount of oil is present in the starter based on comparing predetermined temperature operational values to the oil temperature.

### BACKGROUND

1. Field

The present disclosure relates to turbomachines, more specifically starters for turbomachines with self-contained oil.

2. Description of Related Art

Some turbomachine starter systems can start a turbomachine cycle by accepting an airflow or exhaust flow from an auxiliary power unit and converting this airflow into rotational motion. The turbomachine is connected to the starter such that the rotation of the starter causes the shaft of the turbomachine to rotate.

Such a starter can include lubricating starter oil within the housing thereof such that the mechanics within the starter can be lubricated. Leaks in the starter and/or general oil consumption can result in a low oil level. As a precaution, the starter must be serviced after a certain amount usage to check and/or replace the oil in the starter.

Such conventional methods and systems have generally been considered satisfactory for their intended purpose. However, there is still a need in the art for starter systems having longer service intervals. The present disclosure provides a solution for this need.

### SUMMARY

In at least one aspect of this disclosure, a starter configured for use with a turbomachine includes a housing defining a cavity, wherein the cavity is configured to hold a quantity of starter oil, and a temperature sensor disposed within the cavity and configured to sense a temperature within the housing.

The temperature sensor can include a thermocouple, thermistor, or any suitable type of sensor. The temperature sensor can be disposed in the housing such that the temperature sensor contacts the starter oil when at least a portion of the starter oil is present in the housing. For example, the temperature sensor can be disposed in or near a sump of the housing.

The starter can further include a speed sensor for sensing a rotational speed of at least one rotating component of the starter, and the temperature sensor can be disposed on the speed sensor within the cavity.

The speed sensor and the temperature sensor can be configured to be connected to a common computing apparatus that is configured to receive and/or interpret signals from the speed sensor and the temperature sensor.

In at least one aspect of this disclosure, a system includes a starter for a turbomachine as described herein, and a computing apparatus configured to receive signals from the temperature sensor and to determine if the temperature within the housing indicates a sufficient quantity of starter oil.

In at least one aspect of this disclosure, a method for determining oil quantity in a starter includes receiving a temperature signal from a temperature sensor disposed within the starter, determining an oil temperature within the starter based on comparing the temperature signal, and determining if a sufficient amount of oil is present in the starter based on comparing predetermined temperature operational values to the oil temperature.

The method can further include activating an alarm to notify a user that the oil quantity is low if the oil temperature is above a predetermined threshold value.

The method can further include estimating the oil quantity in the starter by correlating the oil temperature to oil quantities associated with the predetermined temperature operational values.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a cross-sectional, side-elevation view of portions of a starter in accordance with this disclosure having a temperature sensor disposed therein;

FIG. 2 is a close up view of the starter of FIG. 1, showing the temperature sensor disposed in a speed sensor; and

FIG. 3 is a cross-sectional front-elevation view of the starter of FIG. 1, showing the position of the speed and temperature sensors.

### DETAILED DESCRIPTION

Reference will now be made to the drawings wherein like reference numerals identify similar structural features or aspects of the subject disclosure. For purposes of explanation and illustration, and not limitation, a schematic view of an embodiment of a starter in accordance with the disclosure is shown in FIG. 1 and is designated generally by reference character 100. The devices, systems, and methods described herein can be used to allow for greater longevity between service intervals of a starter used to start an engine (e.g., a gas turbine engine).

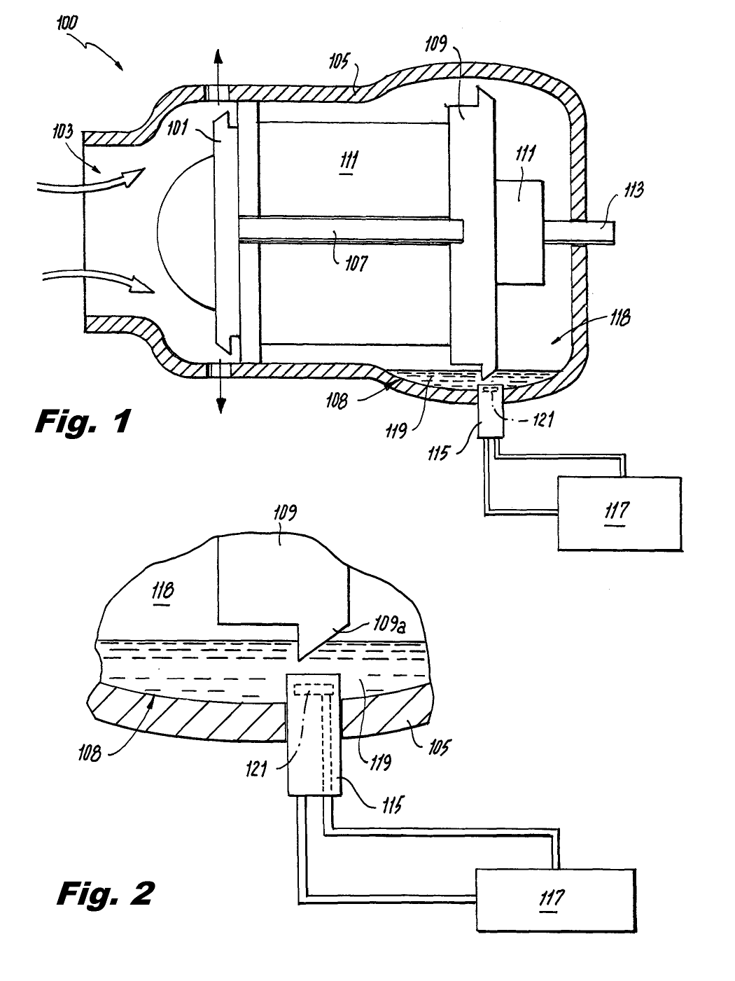

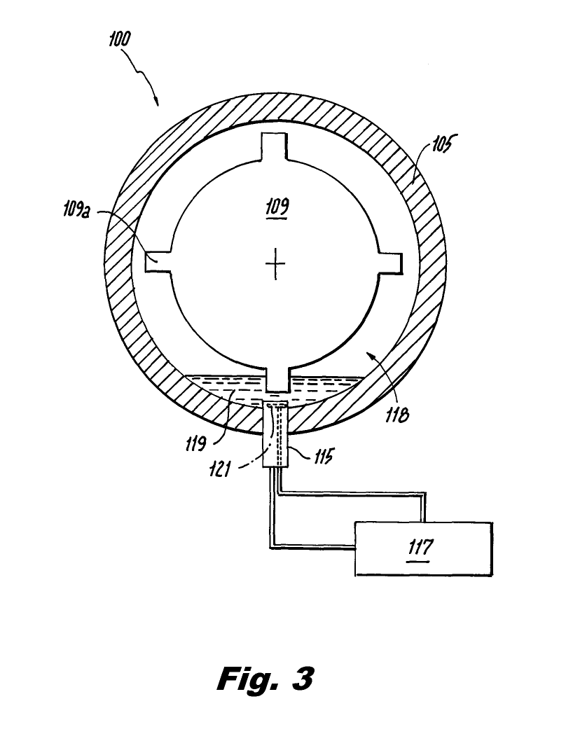

In at least one aspect of this disclosure, referring to FIGS. 1-3, a starter 100 configured for use with a turbomachine includes a housing 105 defining a cavity, wherein the cavity 118 is configured to hold a quantity of starter oil 119, and a temperature sensor 121 disposed within the cavity 118 and for sensing the temperature within the housing 105.

The starter 100 includes a turbine 101 for converting fluid flow, e.g., a gas flow, received from an inlet 103 to rotational energy. Starter 100 can further include a shaft 107 connected for common rotation with the turbine 101 and to one or more mechanical components within the housing 105. For example, the shaft 107 can be connected to a gear 109 (e.g., a ring gear) which can include speed pickup gear teeth 109a. Gear 109 can be connected to any other suitable transmission 111 and directly or indirectly to an output shaft 113 configured to link to an engine. In certain embodiments, the air inlet 103 is configured to accept an airflow from an auxiliary power unit (APU) of an aircraft and the output shaft 113 is configured to attach to a turbomachine to start the turbomachine rotation, e.g., to start a gas turbine engine on an aircraft.

The temperature sensor 121 can include and suitable device configured to sense temperature, such as, but not limited to, a thermocouple. In some embodiments, the temperature sensor 121 can be disposed in the housing 105 such that the temperature sensor 121 is in thermal and/or physical contact with the starter oil 119 when at least a portion of the starter oil 119 is present in the housing 105. For example, the temperature sensor 121 can be disposed within oil sump 108 that forms part of cavity 118. The temperature sensor 121 can otherwise be disposed in thermal communication with any suitable portion of the starter 100 that directly or indirectly indicates the temperature of starter oil 119.

During operation, the starter oil 119 can be configured to circulate/whip around within the cavity 118. In such embodiments, the temperature sensor 121 can be disposed in housing 105 in any suitable location where the oil flows.

The starter 100 can further include a speed sensor 115 for sensing a rotational speed of at least one rotating component of the starter 100. In certain embodiments, the speed sensor 115 is configured to determine the speed of speed pickup gear teeth 109a of gear 109 as gear 109 rotates.

The temperature sensor 121 can be disposed on the speed sensor 115 within the cavity 118 as shown in FIGS. 1-3. In at least some embodiments, the speed sensor 115 and the temperature sensor 121 are configured to be connected to a common computing apparatus 117 that is configured to receive and/or interpret signals from the speed sensor 115 and the temperature sensor 121. The common computing apparatus 117 can include an electronic engine control (EEC) system.

For example, a speed sensor 115 can be retrofitted to include the temperature sensor 121 such that additional electronics and computing components are not needed. In such embodiments, the common computing apparatus 117 can include retrofitted software and/or hardware that is configured to receive, interpret, and/or otherwise process signals from the temperature sensor 121 to determine the temperature of the oil and output a signal (e.g., a signal indicating oil level displayed to a user, an alarm signal if temperature is too high or low). The common computing apparatus 117 can be configured to determine if the oil level is too low and/or too high (e.g., from accidental overfill). For example, an abnormally increased temperature can be indicative of low oil and/or of high oil. Also, the common computing apparatus 117 can correlate a sensed rotational speed to expected temperatures for a suitable amount of oil to determine if the oil quantity is sufficient for operation.

In at least one aspect of this disclosure, a system includes a starter 100 as disclosed herein configured for use with a turbomachine, and a computing apparatus 117 configured to receive signals from the temperature sensor 121 and to determine if the temperature within the housing 105 indicates a sufficient quantity of starter oil 119. The temperature sensor 121 is integrated into an EEC system that includes one or more speed sensors 115 and a common computing apparatus 117.

In at least one aspect of this disclosure, a method for determining oil quantity in a starter 100 includes receiving a temperature signal from a temperature sensor 121 disposed within the starter 100, determining an oil temperature within the starter 100 based on the temperature signal, and determining if a sufficient amount of oil 119 is present in the starter 100 based on comparing the predetermined temperature operational values to the oil temperature.

The method can further include activating an alarm to notify a user that the oil quantity is low if the oil temperature is above a predetermined threshold value (e.g., about 250 degrees F.). The method can include estimating the oil quantity in the starter 100 by correlating the oil temperature to oil quantities associated with the predetermined temperature operational values.

In some embodiments, the signal received from the temperature sensor 121 need not be interpreted and/or converted to a temperature signal, and the signals electrical characteristics (e.g., voltage, resistance, impedance, current) can be correlated directly to predetermined data that associates such signal characteristics to an oil quantity. In such embodiments, the method can further include activating an alarm to notify a user that the oil quantity is low if the signal characteristics are outside a threshold value for the respective characteristic (e.g., impedance is too high or low).

The herein described methods can be implemented using any suitable electronic hardware (e.g., circuits, processors, memory, etc.), software (e.g., any suitable programming language), or the like such that the steps herein can be automated.

The devices, methods, and systems of the present disclosure, as described above and shown in the drawings, provide for an engine starter (e.g., a pneumatic starter for a turbomachine) with superior properties including increased time between servicing intervals. While the apparatus and methods of the subject disclosure have been shown and described with reference to embodiments, those skilled in the art will readily appreciate that changes and/or modifications may be made thereto without departing from the spirit and scope of the subject disclosure.

What is claimed is:

1. A starter configured for use with a turbomachine, comprising:
a housing defining a cavity, wherein the cavity is configured to hold a quantity of starter oil; and
a temperature sensor disposed within the cavity and configured to sense a temperature within the housing.

2. The starter of claim 1, wherein the temperature sensor includes a thermocouple.

3. The starter of claim 1, wherein the temperature sensor is disposed in the housing such that the temperature sensor contacts the starter oil when at least a portion of the starter oil is present in the housing.

4. The starter of claim 1, wherein the starter further includes a speed sensor for sensing a rotational speed of at least one rotating component of the starter, wherein the temperature sensor is disposed on the speed sensor within the cavity.

5. The starter of claim 4, wherein the speed sensor and the temperature sensor are configured to be connected to a common computing apparatus that is configured to receive and/or interpret signals from the speed sensor and the temperature sensor.

6. A system, comprising:
a starter configured for use with a turbomachine, comprising
a housing defining a cavity, wherein the cavity is configured to hold a quantity of starter oil; and
a temperature sensor disposed within the cavity and configured to sense a temperature within the housing; and
a computing apparatus configured to receive signals from the temperature sensor and to determine if the temperature within the housing indicates a sufficient quantity of starter oil.

7. The system of claim 6, wherein the temperature sensor includes a thermocouple.

8. The system of claim 6, wherein the temperature sensor is disposed in the housing such that the temperature sensor contacts the starter oil when at least a portion of the starter oil is present in the housing.

9. The system of claim 6, wherein the starter further includes a speed sensor for sensing a rotational speed of at least one rotating component of the starter, wherein the temperature sensor is disposed on the speed sensor within the cavity.

10. The starter of claim 9, wherein the speed sensor is connected to the computing apparatus such that the computing apparatus is configured to receive and/or interpret signals from the speed sensor and the temperature sensor.

11. A method for determining oil quantity in a starter, comprising:
receiving a temperature signal from a temperature sensor disposed within the starter;
determining an oil temperature within the starter based on the temperature signal; and
determining if a sufficient amount of oil is present in the starter based comparing the oil temperature to the predetermined temperature operational values.

12. The method of claim 11, further including activating an alarm to notify a user that the oil quantity is low if the oil temperature is above a predetermined threshold value.

13. The method of claim 12, further including estimating the oil quantity in the starter by correlating the oil temperature to oil quantities associated with the predetermined temperature operational values.
